(19) United States
(12) Patent Application Publication
(10) Pub. No.: US 2010/0119608 A1
(43) Pub. Date: May 13, 2010

(54) Synthesis of pH-sensitive, Acid-Stable Metal-Binding Nanoparticles

(76) Inventor: Jinhyuk Fred Chung, Daejon (KR)

Correspondence Address: FENWICK & WEST LLP, SILICON VALLEY CENTER, 801 CALIFORNIA STREET, MOUNTAIN VIEW, CA, 94041, US

(21) Appl. No.: 12/268,977

(22) Filed: Nov. 11, 2008

(51) Int. Cl. A61K 9/14; A61K 31/295; A61K 31/315; A61K 31/28; A61K 31/19

(52) U.S. Cl. 424/489; 514/502; 514/494; 514/495; 514/574; 977/906

(57) ABSTRACT

Among natural mechanisms of cell deaths, disease or toxicity, one of the most common method is through overloading of certain biological metals such as calcium, iron and zinc. We propose to utilize this natural mechanism of cell death against cancer by utilizing the well known phenomenon of enhanced permeation and retention effect (EPR effect) and metal-binding nanoparticle moieties that can self-degrade under certain biological conditions such as pH. More specifically, we show that one can form nanoparticles that consist of polymerized citric acid and various different types of metals including, but not limited to, iron, calcium, zinc, silver and magnesium, displaying acid-stability and self-degradation leading to constituent metal release when pH rises closer to the neutral pH of 7 or higher. We also show that these nanoparticles with different metal compositions have distinct cytotoxicity against various different types of cancer cell lines, including B16F10 melanoma, H460 human lung cancer, T98G kidney cancer, Ramos leukemic cancer, etc. in vitro. We also show evidence of in vivo anti-cancer activity of our nanoparticles containing various different metals using mouse model studies.

### CROSS-REFERENCE TO RELATED APPLICATIONS

This application is a continuation-in-part of and claims priority to U.S. patent application Ser. No. 12/267,772, filed Nov. 10, 2008, which application is incorporated herein in its entirety by the reference thereto.

### FIELD OF INVENTION

The present invention relates to synthesis of organometallic nanoparticles and their use as target deliver systems. More particularly, the invention relates to nanoparticles comprising chelating organic acids and biologically active metals.

### BACKGROUND

Nanoparticles, having a typical diameter in the range of 1 to 1000 nm, have been used as catalysts, photocatalysts, adsorbents, and sensors. Recently, nanoparticles have been used for the treatment of diseases. Nanoparticles can bind or be linked to natural or synthetic substances such as drugs, medicaments, diagnostic agents, antisense oligonucleotides, proteins, plasmids etc. and carry such substances to target organs in the human or animal body, such as the brain, liver, kidneys and other organs (WO 95/22963 and WO 98/56361).

In particular, nanoparticles have been used for the treatment of cancers. Nanoparticles conjugated to drugs can be delivered to specific sites by either active targeting or by size-dependant passive targeting (Cancer Res. 1986; 46:6387-6392; J. Control. Release 1999; 62:253-262).

Active drug targeting is a method of selectively delivering anticancer elements to cancer cells by conjugating nanoparticles containing anticancer agents to recognition groups that bind or react with cancer cells. Nanoparticles-based drugs designed in this manner allow for controlled local release of drugs at specific drug targets defined by the recognition groups. Prime examples of active targeting method are lectin and carbohydrate, ligand and acceptor, or antibody and antigen (Farhan J. Ahmad, et al., Nanotechnology: A Revolution in the Making, The Pharma Review December 2005).

Lectin and carbohydrate binding is one of the conventional methods for specific drug delivery system. Lectin is a nonimmune protein that can recognize and bind with glycoprotein on the surfaces of cells. Interaction between lectin and a specified carbohydrate is achieved very specifically. Therefore, a carbohydrate moiety is used to bind the drug delivery system to lectin (direct lectin targeting), and the lectin can be used again as a targeting moiety to bind with a carbohydrate on the surface of a targeting cell (reverse lectin targeting).

Passive drug targeting, on the other hand, employs enhanced permeation and retention (EPR) effect to specifically target cancer cells. The EPR effect is a phenomenon which appears widely only in cancer cells and in angiogenic vascular structures of cancer. The EPR effect in cancer cells is characterized by non-selective absorption, permeation, and retention of macromolecules having a macromolecule size between 10 to 100 nm.

The passive targeting strategy has advantages over the active targeting strategy. First advantage is that passive designs do not require recognition groups, making production procedures and drug administration relatively simple. The second advantage is that the lack of specificity allows for general application of the passively designed anti-cancer drugs to any cancer types displaying EPR effect. Lastly, the consequent ability of such drugs to accumulate in cancer that come in contact with blood supply makes it easier to combat highly metastatic or mutating cancer.

Recently, passive targeting strategy has been employed against cancer cells by conjugating nanoparticles with conventional anti-cancer toxins such as radioactive/toxic heavy metals (Gadolinium, Holmium-166, Copper) or cytotoxins (FU-5) (H. Tokumitsu, et al., Chitosan-gadopentetic acid complex nanoparticles for gadolinium neutron capture therapy of cancer: preparation by novel emulsion droplet coalescence technique and characterization, Pharm. Res. (1999) 16: 1830-1835; Kim J K, et al., Long-term clinical outcome of phase IIb clinical trial of percutaneous injection with holmium-166/chitosan complexes (Milicam) for the treatment of small hepatocellular carcinoma Clin Cancer Res. (2006) 12(2): 543-8; Qi L, et al., Cytotoxic activities of chitosan nanoparticle and copper-loaded nanoparticles, Bioorg Med Chem Lett. (2005) 15(5): 1397-9). These studies showed selective effectiveness of the conjugated toxins against cancers through enhanced necrosis, growth inhibition, and reduced metastasis when compared to those that were not fused with nanoparticles, as well as a reduction in side effects. However, the conjugated nanoparticles were shown to accumulate in brain/spinal cord/bone marrow (K. Ringe, et al. Nanoparticle Drug Delivery to the Brain, Encyclopedia of Nanotechnology (2004) volume 7: pages 91-104), resulting in critical toxicity and side effects in the respective organs that result from slow release of the toxic conjugates.

Therefore, there is a need for the development of anticancer drugs having wide and effective anticancer effect without short or long-term toxicity in order to effectively combat cancer. The present invention meets the need, and discloses water-soluble organometallic nanoparticles with biologically active metals with little or no toxicity.

### SUMMARY

The present invention provides compositions of and methods for synthesizing nanoparticles and the use of the nanoparticles for the treatment or prevention of cancer. The nanoparticles of the invention comprise pH-sensitive, self-dissociating organometallic nanoparticles that are stable under acidic conditions and that dissociate near neutral pH or under physiological conditions.

In one aspect, the invention provides a water-soluble nanoparticle, the nanoparticle comprising an organic compound can form an ester and can chelate a metal. The nanoparticles is stable under acidic conditions and dissociates near neutral pH. The organic compound can be the organic compound is citric acid, isocitric acid, glutamic acid, 3-aminopentanedioic acid or combinations thereof.

In one aspect, the invention provides a water-soluble nanoparticle, the nanoparticle comprising an organic compound of formula I

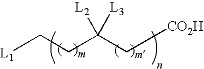

wherein L₁, L₂, and L₃ are independently selected to be H, OH, halogen, NR₁R₂, SH, SO₃R₃, or CO₂R₄, wherein R₁, R₂, R₃, and R₄ can independently be H or lower alkyl, and m, m′, and n can be independently selected to be an integer between 0 and 20; and a metal and/or a metal salt wherein the nanoparticle has a size between about 50 nm to about 500 nm.

In another aspect of the invention, methods of producing nanoparticles are provided, the method comprising combining an organic compound of formula I

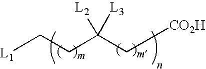

wherein L₁, L₂, and L₃ are independently selected to be H, OH, halogen, NR₁R₂, SH, SO₃R₃, or CO₂R₄, wherein R₁, R₂, R₃, and R₄ can independently be H or lower alkyl, and m, m′, and n can be independently selected to be an integer between 0 and 20; a metal or a metal salt to provide a reaction solution; and stirring the reaction solution to provide the nanoparticles.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates the transmission electron microscope (TEM) images of the organometallic nanoparticles (OFeCa-1). FIG. 1A is the TEM image at 5×10³ magnification. FIG. 1B is the TEM image at 4.3×10⁴ magnification. FIG. 1C is the TEM image at 4.5×10⁵ magnification.

FIG. 2 illustrates the stability of OFeCa-1 as a function of pH.

FIG. 3 illustrates the results of the dynamic light scattering (DLS) as a function of pH.

FIG. 4 illustrates the results of dialysis using a 1000 Da membrane as a function of pH.

FIG. 5 illustrates the effect of OFeCa-1 on various cancer cell lines.

FIG. 6 illustrates the effect of OFeCa-1 on mouse model bearing B16F10 mouse melanoma in the lungs.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York, and Cotton et al. (1999) “Advanced Inorganic Chemistry 6^{th} Ed.” Wiley, N.Y.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiologically acceptable range” or “near neutral pH” is meant a pH in the range of approximately 6.0 to 8.0 inclusive, more typically in the range of approximately 6.5 to 7.6 inclusive.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

### II. Overview

The present invention discloses nanoparticles, compositions of nanoparticles and methods for synthesizing nanoparticles and the use of the nanoparticles for the treatment or prevention of cancer. The nanoparticles of the invention comprise pH-sensitive, self-dissociating organometallic nanoparticles. The nanoparticles are stable under acidic conditions and dissociate near neutral pH or under physiological conditions. The nanoparticles of the invention have a dissociation rate such that majority of the dissociate occurs in the cancer cells and not in the circulatory system, and their dissociation in the cells releases metal in high enough concentration to kill the cancer cell. The metal of the nanoparticle can be selected such that it has cytotoxic properties, either alone or in combination with other drugs, but is not toxic to the normal cells at the therapeutic concentrations.

Thus, the nanoparticles of the invention are water-soluble organometallic nanoparticles comprising an organic molecule that can form an ester and that can bind metal and/or metal ions. The nanoparticles can be used for the treatment or prevention of a cancer. Without being bound to a theory, the nanoparticles of the invention act as anticancer agents via the enhanced permeation and retention (EPR) effect.

Nanoparticles of the present invention can be retained selectively in cancer cells via the EPR effect. And upon sufficient accumulation the nanoparticles expose the cancer cells to direct/indirect oxidative stress, causing specific toxicity at site. The nanoparticles of the present invention having such anticancer mechanism consist of a multidentate metal chelating organic compound and a plurality of minerals, some of which are capable of generating direct oxidative stress to the cancer cells, and others with biological functionality to trigger biological reactions/cycles/cascades.

### II. Organic Molecule

In one aspect of the invention, the nanoparticles comprise an organic molecule. The organic molecule can be selected such that it can form an ester and it can bind or chelate metal and/or metal ions. Thus, the organic molecule can have the following structure

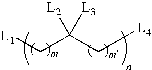

wherein L₁, L₂, L₃ and L₄ can be independently selected to be H, OH, halogen, NR₁R₂, SH, SO₃R₃, and CO₂R₄, wherein R₁, R₂, R₃, and R₄ can independently be H or lower alkyl, and where at least one of L₁, L₂, L₃, or L₄ can form an ester, and m, m′, and n can be independently selected to be an integer between 0 and 20.

Preferably, the organic molecule can have structure of formula I:

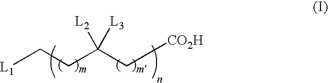

wherein L₁, L₂, and L₃ can be independently selected to be H, OH, halogen, NR₁R₂, SH, SO₃R₃, and CO₂R₄, wherein R₁, R₂, R₃, and R₄ can independently be H or lower alkyl, and m, m′, and n can be independently selected to be an integer between 0 and 20.

Exemplary organic molecules include diethylenetriaminepentaacetic acid (DTPA), ethylenedinitrilotetraacetic acid (EDTA), N,N-bis(carboxymethyl)glycine (NTA), 2,3-bis-sulfanylpropanoic acid, as well as compounds having the following structures:

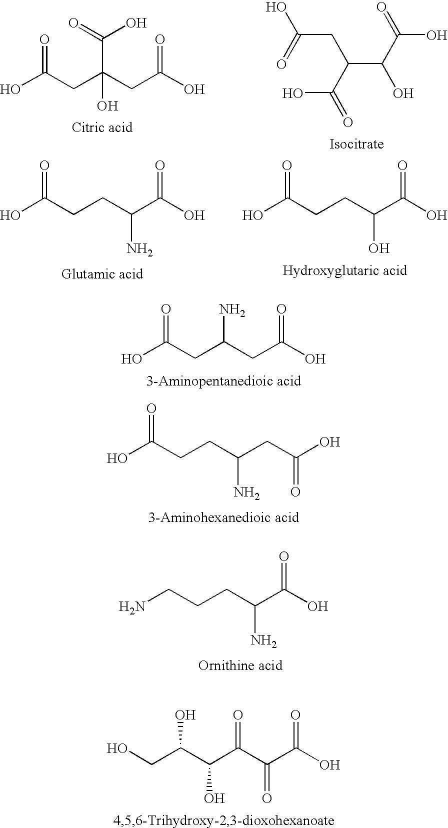

Preferably, the organic acid is a biological compound, such as citric acid.

### III. Metal

The metal for use in the nanoparticles of the present invention can be selected such that the metal has low toxicity to normal cells at the therapeutically effective dose, can be easily removed/detoxified from the body, and can cause oxidation or other stress in cells. Thus, the metal or the metal ion for use in the nanoparticles of the invention can be selected to include iron, magnesium, manganese, titanium, cesium, silver, gold, platinum, nickel, or combinations thereof, and can also include minerals such as calcium, zinc, potassium or sodium that have no inherent toxicity but are capable of causing indirect oxidation stress by triggering biological reactions/cycles/cascades in any cell upon significant accumulation and steady release.

The metals for use in this invention include the Group 1A, 2A, 3A, 7A, 8A, 1B, 2B, 3B, 4B, 5B, 6B, 7B and 8B elements. Preferred metals include those selected from the group consisting of Na, K, Cs, Mg, Ca, Ba, Ti, Zr, Mn, Fe, Co, Ni, Ru, Rh, Pd, Pt, Cu, Ag, Au, Zn, Cd, Si, Sn, or combinations thereof. More preferably, the metal is Fe, Ca, Mg, Mn, K, Na, Zn, Ti, Si, Cs, Cu, Ag, Au, Pt, Ni or combinations thereof.

The metal or the metal ion can be selected from a Group V metal, such as V or Nb, and mixtures thereof, a Group VI metal including Cr, W, or Mo, and mixtures thereof, VII metal, such as, Mn, or Re, Group VIII metal including Co, Ni, Ru, Rh, Pd, Os, Ir, Pt, and mixtures thereof, or the lanthanides, such as Ce, Eu, Er, or Yb and mixtures thereof, or transition metals such as Cu, Ag, Au, Zn, Cd, Sc, Y, or La and mixtures thereof. Specific examples of mixture of metals, such as bimetallic metals, which may be employed by the present invention include Fe—Pt, Fe—Mo, Ni—Fe—Mo, Co—Cr, Co—W, Co—Mo, Ni—Cr, Ni—W, Ni—Mo, Ru—Cr, Ru—W, Ru—Mo, Rh—Cr, Rh—W, Rh—Mo, Pd—Cr, Pd—W, Pd—Mo, Ir—Cr, Pt—Cr, Pt—W, and Pt—Mo.

In one aspect of the invention, the metal can be iron. Without being bound to theory, it is thought that iron functions as an anticancer agent via superoxide. For example, the application of iron to a cell through liposome in a cationic porphyrin form (Yuasa M., et al., Liposomal surface-loading of water-soluble cationic iron (ITT) porphyrins as anticancer drugs, Mol Pharm. (2004) 1(5): 387-9) was theorized to produce superoxide in cancer cells. In the case of calcium, calcium cascade is among the most powerful and well known biological reactions that trigger numerous other biological activities, and hence the effect of over-accumulating calcium in cancer cells in our proposed manner is likely to be effective. In fact, similar mechanism of toxicity and cell death occur in neuronal cells of neurodegenerative diseases where increased level of in tracellular calcium is the main cause of stress-induced cell death in the respective cells (Van Damme P. et al., Excitotoxicity and amyotrophic lateral sclerosis, Neurodegener Dis. (2005) 2 (3-4): 147-59).

### IV. Synthesis

The metal, the metal ion, the bimetal, or combination of metals can be used to prepare organometallic nanoparticles having defined particle size and diameter distribution. The nanoparticles can be prepared by incubating the organic compound with the metal or the metal salt. The ratio of the organic compound to the metal salt is not important since the reaction will reach completion when all the chelation sites have been occupied. However, the ratio of the organic compound to the metal can be about 1:10 (M/M) to about 20:1 (M/M), preferably about 1:5 (M/M) to about 15:1 (M/M), and more preferably about 1:1 (M/M) to about 10:1 (M/M). Optionally, the reaction mixture comprising the organic compound and the metal has at least one solid-phase metal source. The solid-phase metal can be mineral iron, zinc, copper, and the like.

The organic compound and the metal can be incubated in the presence or absence of a solvent. If a solvent is added, the solvent is selected such that it does not react with the metal or prevent chelation from occurring. The solvent can thus be an non-reactive organic solvent, such as, for example, hexane, acetone, DMSO, DMF, and the like.

The reaction mixture can be allowed to stir until the reaction reaches completion. Typical reaction times range from about 20 minutes to about 2 months, depending on the desired nanoparticle. The reaction mixture can be stirred at a temperature of about −20° C. to about 100° C., more preferably about 0° C. to about 70° C., even more preferably about 20° C. to about 50° C., and any temperature in between.

The water-soluble organometallic nanoparticles thus produced can have an average particle size of about 0.1 nm to about 20 nm, more preferably about 0.2 nm to about 3 nm and most preferably about 0.3 nm to 2 nm. The nanoparticles can thus have a particle size of 0.1, 1, 2, 3, 4, 5, 6, 7, 8, 9, or 10 nm, and up to about 20 nm. In another aspect, the nanoparticles can have a range of particle size, or diameter distribution. For example, the nanoparticles can have particle sizes in the range of about 0.1 nm and about 5 nm in size, about 3 nm and about 7 nm in size, or about 5 nm and about 11 nm in size.

In one aspect of the invention, the diameter distribution of the synthesized organometallic nanoparticles can be substantially uniform. Thus, about 90% of the nanoparticles have a diameter within about 25% of the mean diameter, more preferably, within about 20% of the mean diameter, and even more preferably, within about 15% of the mean diameter. Thus, the diameter distribution of the nanoparticles can be about 10% to about 25% within the mean diameter, more preferably about 10% to about 20% of the mean diameter, and even more preferably about 10% to about 15% of the mean diameter.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Preparation of the Organometallic Complex (OFeCa-1)

An acidic solution of about 100 g/L (about 470 mM) of citric acid (monohydrate) in water, with a pH of about 1.6, was prepared. In a round bottom flask was placed 200 mL of the acidic solution, 50 g of electrolytic iron metal (Toho Zinc, Tokyo, Japan). The reaction mixture was stirred at 50° C. for 2 weeks. At the end of the incubation, 5.7 g of CaCl₂(hydrate), 3.06 g of MnSO4 (hydrate), and 0.9 g of ZnSO₄ (hydrate) were added and the mixture was stirred at 50° C. for 3 days. The organometallic complex (OFeCa-1) thus obtained was separated by evaporation of the solvent, and recrystalization of the powder using water.

### Example 2

### Preparation of the Organometallic Complex (OFeCa-1)

In a round bottom flask was placed 200 mL of the acidic solution prepared in Example 1, 50 g of electrolytic solid iron metal (Toho Zinc, Tokyo, Japan), 5.7 g of CaCl₂(hydrate), 3.06 g of MnSO4 (hydrate), and 0.9 g of ZnSO₄ (hydrate). The reaction mixture was stirred at 50° C. for 3 weeks. The organometallic complex (OFeCa-1) thus obtained was separated by evaporation of the solvent, and recrystalization of the powder using water.

### Example 3

### Characterization of OFeCa-1

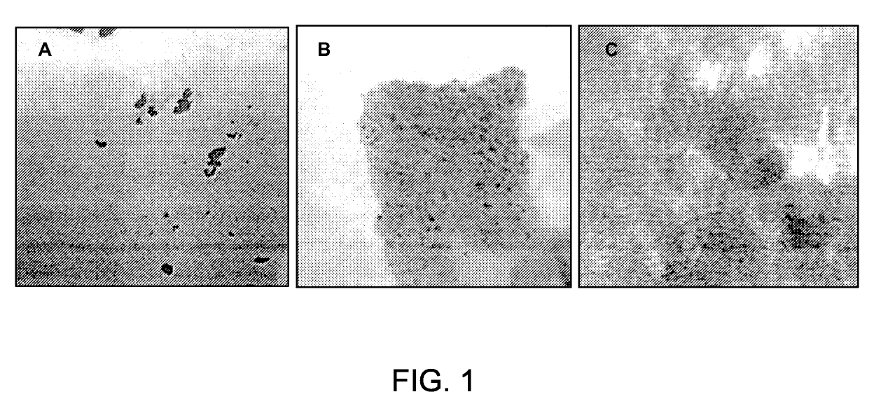

The organometallic complex (OFeCa-1) prepared in Examples 1 and 2 were characterized using transmission electron microscopy (TEM). The TEM images are given in FIGS. 1A to 1C. FIG. 1A shows OFeCa-1 forms amorphous and polydisperse nanoparticles of about 50 nm to about 500 nm. Each organometallic nanoparticles appears to consist of smaller nanometal particles of about 6-20 nm (FIG. 1B) having both crystalline and non-crystalline metal containing moieties (FIG. 1C).

The metal content of the OFeCa-1 was determined using ICP-MS and atomic absorption.

| Metal | ppm | dry % |
|---|---|---|
| Fe | 10425.20 | 9.48 |
| Ca | 7086.86 | 6.44 |
| Mg | 2572.65 | 2.34 |
| Zn | 923.95 | 0.84 |
| Mn | 250.40 | 0.23 |
| Cu | 10.41 | 0.01 |
| Ni | 1.66 | 0.00 |

The nanoparticles contain iron, calcium, zinc, and magnesium as the major components.

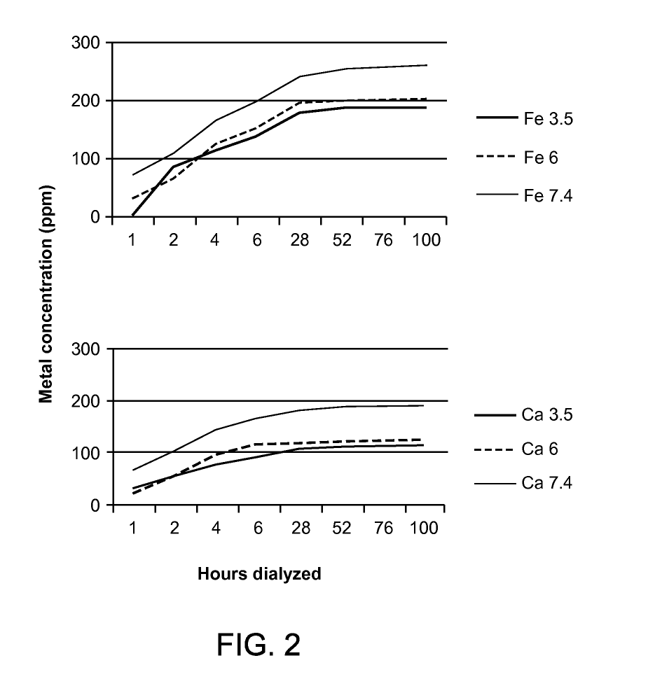

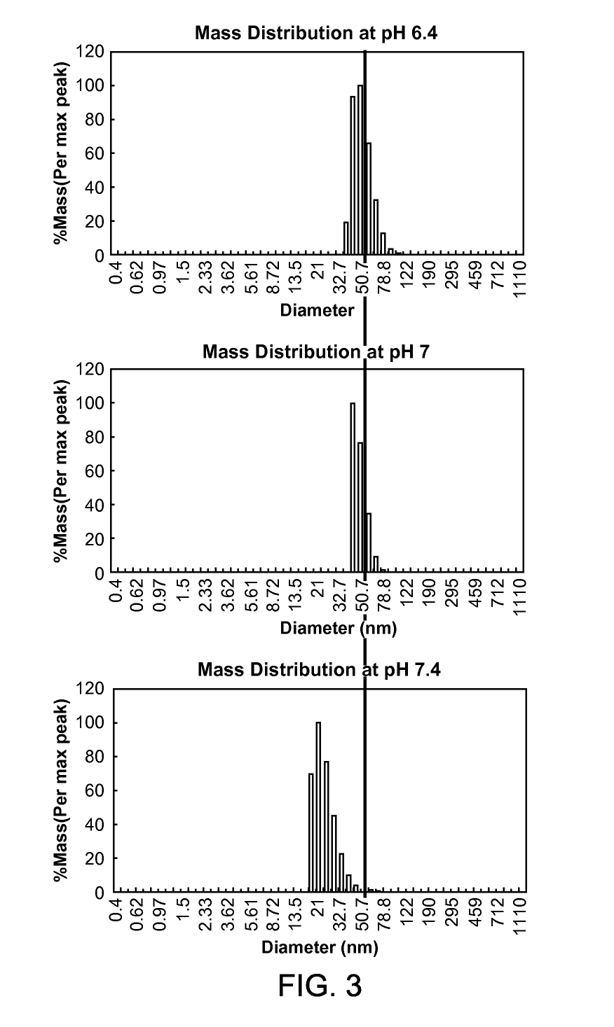

The stability of OFeCa-1 as a function of pH was determined using dynamic light scattering (DLS) and dialysis. DLS showed incremental reduction in the diameter of the nanoparticles as pH was increased from pH 3 to pH 7.4 (FIG. 2). The results are consistent with the OFeCa-1 nanoparticles being stable at acidic pH and degrading at neutral pH. The time dependent release of the degraded metal components of the OFeCa-1 organometallic nanoparticles were determined using dialysis experiments conducted using membranes with molecular weight cut-offs at 1000 Da. The kinetics of the release of the metal are shown in FIG. 3, and support the conclusions from the DLS experiment. The dialysis experiments show a faster metal release kinetics at higher pH. Thus, the OFeCa-1 organometallic nanoparticles are stable at acidic pH and dissociate to release the metal near the physiological pH.

### Example 4

### Effectiveness of OFeCa-1 Against Cancer Cell Lines

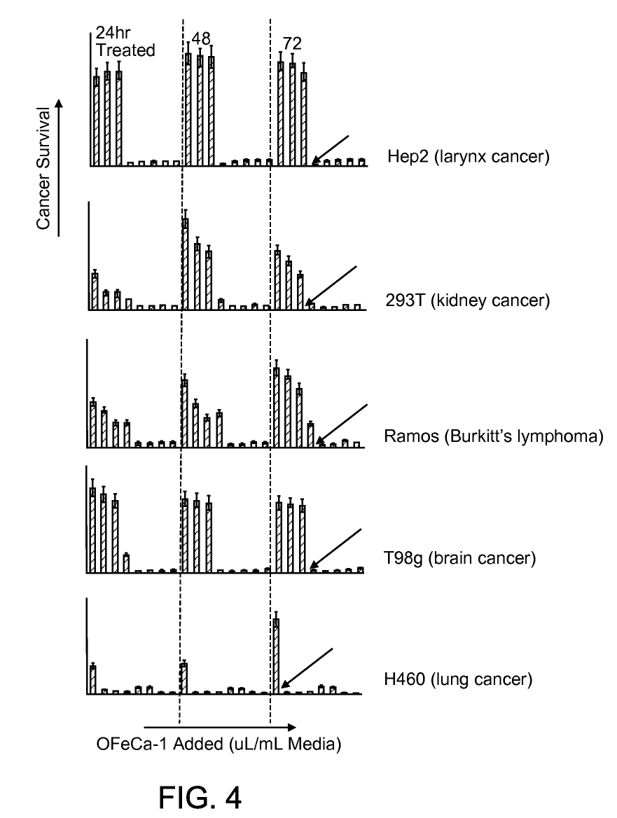

The organometallic complex (OFeCa-1) prepared in Examples 1 and 2 was tested in vitro against various cancer cell lines, including Heep2 (human larynx), 293T (human kidney), T98g (human brain), H460 (human lung), Ramos (human Burkitt's lymphoma), and B16F10 (mouse melanoma). The cell lines ere cultured on a dish in the presence or absence of OFeCa-1 and tested using Cell Counting Kit-8™ (Dojindo, Tokyo). The results are illustrated in FIG. 4, and show OFeCa-1 exhibits non-classical dosage dependence curve where OFeCa-1 cirtically effective upon reaching a concentration of about 20 μL of 11% w/w OFeCa-1 solution per 1 mL of growth media.

### Example 5

### Effectiveness of OFeCa-1 Against Mouse Models

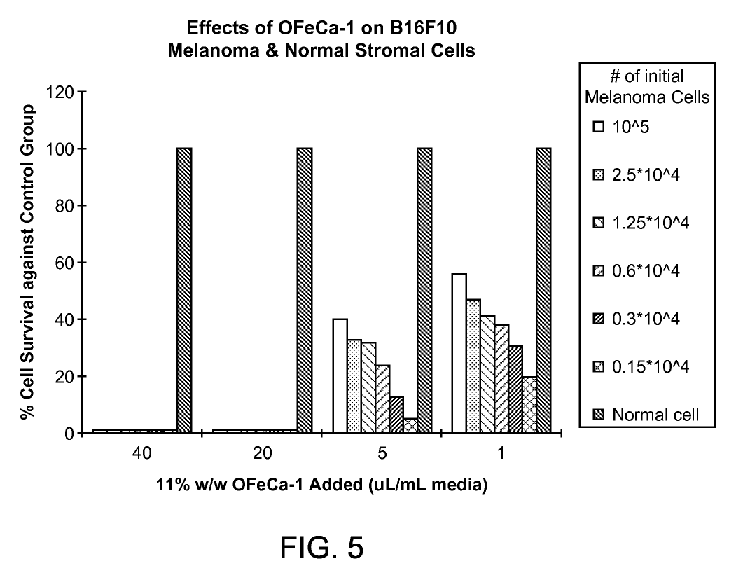

The organometallic complex (OFeCa-1) prepared in Examples 1 and 2 was tested in vivo against c57B mice bearing B16F10 mouse melanoma in lungs. The mice were divided into two experimental groups and a control group. The first experimental group was treated with OFeCa-1 and then injected with 100,000 B16F10 melanoma cells suspended in PBS buffer. The second experimental group was injected with 100,000 B16F10 melanoma cells suspended in PBS buffer and 5 days later treated with OFeCa-1. The results, illustrated in FIG. 5, show dose dependant effect of OFeCa-1, and show that OFeCa-1 is effective in treating melanoma.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

We claim:

1. A water-soluble nanoparticle, the nanoparticle comprising an organic compound of formula I 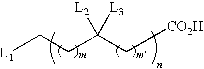
wherein L₁, L₂, and L₃ are independently selected to be H, OH, halogen, NR₁R₂, SH, SO₃R₃, or CO₂R₄, wherein R₁, R₂, R₃, and R₄ can independently be H or lower alkyl, and m, m′, and n can be independently selected to be an integer between 0 and 20; and
a metal and/or a metal salt wherein the nanoparticle has a size between about 50 nm to about 500 nm.

2. The nanoparticle of claim 1, wherein the nanoparticle is stable under acidic conditions.

3. The nanoparticle of claim 2, wherein the nanoparticle dissociates near neutral pH.

4. The nanoparticle of claim 1, wherein the organic compound can form an ester and can chelate a metal.

5. The nanoparticle of claim 4, wherein the organic compound is citric acid, isocitric acid, glutamic acid, or 3-aminopentanedioic acid.

6. The nanoparticle of claim 5, wherein the organic compound is citric acid.

7. The nanoparticle of claim 1, wherein the metal is selected from the group consisting of Fe, Ca, Mg, Mn, K, Na, Zn, Ti, Si, Cs, Cu, Ag, Au, Pt, Ni, and combinations thereof.

8. The nanoparticle of claim 7, wherein the metal is Fe, Ca, Zn, Ag, or combination thereof.

9. The nanoparticle of claim 1, wherein at least about 90% of the nanoparticles have a diameter of not more than about 500 nm.

10. The nanoparticle of claim 1, wherein at least about 90% of the nanoparticles have a diameter of not more than about 100 nm.

11. The nanoparticle of claim 1, wherein the nanoparticle has a diameter of about 50 nm to about 500 nm.

12. A method of producing nanoparticles, the method comprising combining an organic compound of formula I 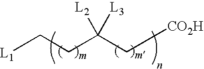
wherein L₁, L₂, and L₃ are independently selected to be H, OH, halogen, NR₁R₂, SH, SO₃R₃, or CO₂R₄, wherein R₁, R₂, R₃, and R₄ can independently be H or lower alkyl, and m, m′, and n can be independently selected to be an integer between 0 and 20;
a metal or a metal salt to provide a reaction solution; and
stirring the reaction solution to provide the nanoparticles.

13. The method of claim 12, wherein the organic compound is citric acid, isocitric acid, glutamic acid, or 3-aminopentanedioic acid.

14. The method of claim 13, wherein the organic compound is citric acid.

15. The method of claim 12, wherein the metal is selected from the group consisting of Fe, Ca, Mg, Mn, K, Na, Zn, Ti, Si, Cs, Cu, Ag, Au, Pt, and Ni.

16. The method of claim 15, wherein the metal is Fe, Ca, Zn, Ag, or combination thereof.
